(19) United States
(12) Patent Application Publication
(10) Pub. No.: US 2010/0274263 A1
(43) Pub. Date: Oct. 28, 2010

(54) Surgical instrument for the placement of ligature clips

(75) Inventors: Alexander Disch, Freiburg (DE); Rupert Mayenberger, Rielasingen (DE)

Correspondence Address: Lipsitz & McAllister, LLC, 755 MAIN STREET, MONROE, CT, 06468, US

(73) Assignee: Aesculap AG, Tuttlingen (DE)

(21) Appl. No.: 12/798,553

(22) Filed: Apr. 5, 2010

(30) Foreign Application Priority Data
Apr. 24, 2009 (DE) 10 2009 018 818.5

(51) Int. Cl. A61B 17/10

(52) U.S. Cl. 606/142

(57) ABSTRACT

In order to maximise the pivoting range of the arms in a surgical instrument for the placement of C-shaped ligature clips having two legs connected to one another by means of a bridge section with two clamping jaws, which in an open position receive an open ligature clip between them and are movable into a closed position by approaching one another and in so doing close the ligature clips by their legs approaching one another, with two elastically deformable arms, each bearing a clamping jaw at its distal end, and with a slide, which is displaceable relative to the arms in the longitudinal direction thereof, and which abuts against the abutment surfaces of the arms and as a result of this abutment against the abutment surfaces elastically pivots the arms relative to one another in a pivoting plane out of a starting position into an end position upon displacement of the slide relative to the arms, in association with a small size of the instrument, it is proposed that at least over a part of their length and at least over a part of their width the two arms are arranged in planes running parallel to the pivoting plane and displaced relative to one another and overlap one another at least partially.

This application claims the benefit of German Patent Application No. 10 2009 018 818.5 filed on Apr. 24, 2009.

The present disclosure relates to the subject matter disclosed in German patent application No. 10 2009 018 818.5 of Apr. 24, 2009, which is incorporated herein by reference in its entirety and for all purposes.

### BACKGROUND OF THE INVENTION

The invention relates to a surgical instrument for the placement of C-shaped ligature clips having two legs connected to one another by means of a bridge section with two clamping jaws, which in an open position receive an open ligature clip between them and are movable into a closed position by approaching one another and in so doing close the ligature clips by their legs approaching one another, with two elastically deformable arms, each bearing a clamping jaw at its distal end, and with a slide, which is displaceable relative to the arms in the longitudinal direction thereof, and which abuts against the abutment surfaces of the arms and as a result of this abutment against the abutment surfaces elastically pivots the arms relative to one another in a pivoting plane out of a starting position into an end position upon displacement of the slide relative to the arms.

Such surgical instruments are described, for example, in patent documents U.S. Pat. No. 3,777,538 A, DE 30 21 099 A1, DE 696 36 965 T2 or DE 696 34 391 T2. In this case, the arms configured as bending arms are connected to one another in one piece and by means of a slide, which engages over the arms and slides along cam-like abutment surfaces, are pivoted out of the open position into the closed position when the slide and the arms are displaced relative to one another in the longitudinal direction, wherein in all cases the arms move in the same plane, i.e. upon full approach the arms abut against one another.

Surgical instruments of this type are predominantly used for minimally invasive procedures, in which the instruments are inserted through small openings in the body into the body by means of generally tubular trocars, and the space conditions are accordingly very confined. The arms that are located next to one another and can be pivoted relative to one another must move within a very narrow cross-section of a barrel-like instrument part, and this results in the pivoting angle of the arms being restricted. In the pivoted-out state, the arms must not project beyond the contour of a barrel of the instrument. Therefore, the arms, which in the pivoted-in end position abut against one another with their inner surfaces, can only be pivoted over a small angle range, and in some circumstances this is not sufficient to accommodate ligature clips. These ligature clips are to be placed against blood vessels and other body parts and in the open position should have as large a spacing as possible between the adjacent legs, so that they can only be received by the clamping jaws of the instrument when these are spaced sufficiently far apart.

It is an object of the invention to configure a surgical instrument of the above type so that in spite of the very confined space conditions a sufficiently large pivoting movement of the arms is possible to allow the clamping jaws to also move sufficiently far apart from one another for open ligature clips.

### SUMMARY OF THE INVENTION

This object is achieved according to the invention with a surgical instrument of the above-described type in that at least over a part of their length and at least over a part of their width the two arms are arranged in planes running parallel to the pivoting plane and displaced relative to one another and overlap one another at least partially.

Because of arrangement of the arms being arranged in planes displaced relative to one another and the possibility of the arms overlapping as a result of this, the arms can be pivoted in the closed position over the whole of the available cross-section without this pivoting movement being hindered by the respectively opposite arm. This firstly allows a larger pivoting motion of the two arms and secondly enables the width of the arms to be increased in the respective pivoting plane, while still retaining a sufficient pivoting range for the arms. This increase in width leads to an increased bending stability of the arms, and this is necessary for the clamping jaws to apply the high clamping forces and in this way bend-resistant clamping arms can also be provided with confined space conditions.

In a first preferred embodiment it is provided that in the overlap region one of the arms is arranged completely in an upper plane and the other arm in a lower plane.

However, in a modified embodiment it can also be provided that in the overlap region one of the arms has a recess open towards the other arm, into which recess the other arm projects at least partially. The overlapping parts of the two arms are then respectively located in different planes and cannot hinder one another, but both arms can still move over a relatively large pivoting range and also have a larger width in their planes, if desired.

It can be provided that the arms already overlap partially in their starting position, in which the clamping jaws are in the open position.

It is additionally possible that in their end position, in which the clamping jaws are in the closed position, the arms overlap at least in some areas over their entire width, i.e. can fill the whole available space.

The overlap can occur over a part of the length of the arms or also over the entire length of the arms.

It is favourable if the width of the arms in the overlap region is greater than their height. The bending moment of the arms in the pivoting plane is quadratically dependent on the width of the arms and only linearly dependent on the height of the arms, so that a widening greatly increases the bending moment. Because the arms are arranged in different planes and because of the possibility of overlapping, the arms can be wider and, if necessary, be configured with a slightly lower height when the space requirement is very small. Nevertheless, not only is there no danger of a reduction of the bending moment as a result of this, but this can possibly even be increased further, although the available space is not larger than in conventional arms that are pivoted relatively to one another in one plane.

It is favourable if in the starting position the side faces of the arms located on the outside are arranged within a contour with bounding means running parallel to one another over the entire length of the arms with the exception of the distal end region. Thus, the arms are not pivoted outwards in the starting position, but run parallel to one another with their outer surfaces, so that they can be accommodated within an outer contour of an instrument, which is not widened towards the distal end.

In particular, it can be provided that the bounding means is formed by the side walls of a chamber of the slide receiving the arms. Therefore, in this case the slide restricts an elastic bending of the arms, which thus run substantially parallel to one another in the resting position.

In a preferred embodiment of the invention it is provided that at its distal end the slide abuts against outwardly projecting abutment surfaces of the arms, which directly adjoin the clamping jaws in proximal direction. As a result, introduction of the closing forces into the arms occurs at the maximum distance from the proximal connection point of the two arms and directly adjacent to the clamping jaws arranged at the distal end of the arms. It is favourable if the abutment surfaces of the two arms lie in different planes, i.e. at a different height in relation to the pivoting plane of the respective arms.

It is particularly advantageous if the abutment surfaces are respectively arranged at the level of the corresponding arms, i.e. in the pivoting plane of these arms. In this case, “in the plane” means that the abutment surfaces do not run above or below the arms, thus the arms are not angled or bent in relation to their pivoting plane. This also assists in reducing the size of the instrument.

It can be provided that the arms are surrounded on all sides by the slide at least on a part of their length. This surrounding on all sides results in a stabilisation of the instrument and protects the arms that can pivot in different planes against undesirable deformation, e.g. perpendicularly to the pivoting planes.

However, it can also be provided that in the overlap region no part of the slide lies opposite the side faces of the arms located on the inside, so that the arms can make use of the whole of the available cross-section of the instrument when pivoting into the end position without abutting against parts of the slide. The slide can have window-like recesses in this region, and it is also possible that on the outer surface of the arms the slide only extends respectively over the height of the arms and thus gives the respective other arm space for the pivoting movement into its end position.

It is advantageous if in the non-deformed state the arms do not overlap over their entire length. This makes production of the arms easier. An overlap only occurs if these are pivoted relative to one another, whether by installation in the instrument already in the starting position or only when the arms pivot into the end position.

The arms can run next to one another from their proximal connection point to their distal end without intersecting, but it is also possible that the arms intersect between their proximal end and their distal end.

In a preferred embodiment it is provided that the arms and the slide are arranged together inside a tubular barrel with a circular contour, beyond which the clamping jaws likewise do not project in their open position. As a result of this, it is possible to insert the instrument into the body through a tubular access point with the clamping jaws open.

The following description of preferred embodiments of the invention serves for more detailed explanation in association with the drawing.

### BRIEF DESCRIPTION OF THE DRAWINGS

FIG. 1 is a perspective view of the clamping jaw region of a surgical instrument with a ligature clip placed on a blood vessel;

FIG. 2 is a perspective view of the distal end of the surgical instrument of FIG. 1 with a cartridge inserted into the instrument to accommodate ligature clips that can be inserted between the clamping jaws;

FIG. 3 is a view similar to FIG. 2 with the cartridge removed from the instrument;

FIG. 4 is a perspective plan view onto the arms of the instrument bearing clamping jaws in a chamber of a slide for closing the clamping jaws;

FIG. 5 is a view similar to FIG. 4 with arms removed from the slide;

FIG. 6 is a perspective view of the arms bearing the clamping jaws in non-deformed state;

FIG. 7 is a view similar to FIG. 4 in a modified exemplary embodiment of a slider;

FIG. 8 is a sectional view taken along line 8-8 in FIG. 5; and

FIG. 9 is a view similar to FIG. 8 with a modified cross-sectional form of the arms.

### DETAILED DESCRIPTION OF THE INVENTION

The instrument 1 shown in the drawing comprises a tubular barrel 2, of which only the distal end is shown in the drawing. At its proximal end not shown in the drawing, the barrel 2 is connected to a handle, on which activation elements are arranged, by means of which the transmission elements passing through the barrel, e.g. push and pull rods, can be moved, the movement of which can thus be transmitted to the distal end of the barrel 2.

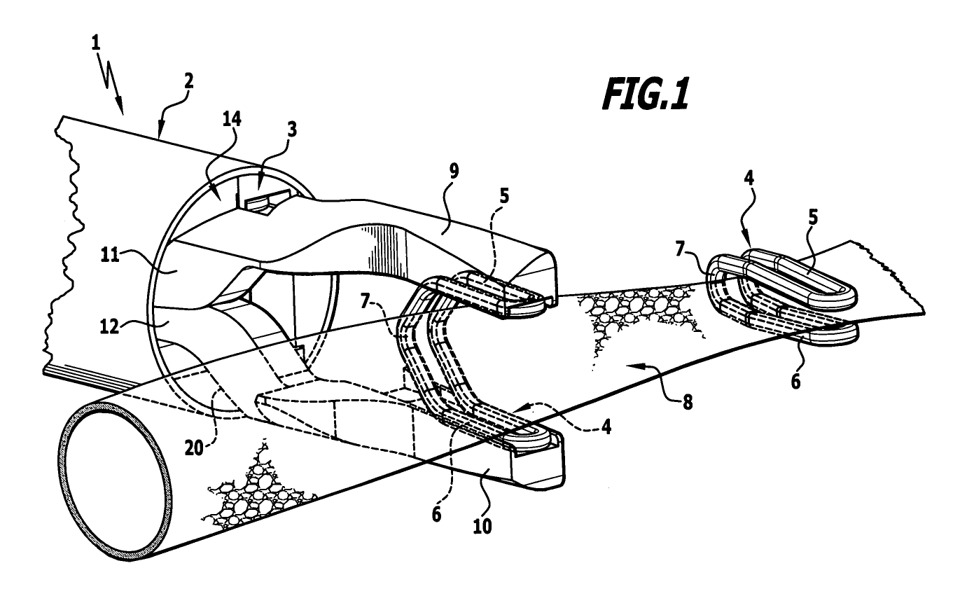

A cartridge 3, which substantially has a semicircular cross-section and in which a plurality of C-shaped ligature clips 4 are arranged, is inserted into the barrel 2 at its distal end. These ligature clips have two legs 5, 6 arranged next to one another and at a spacing from one another, which are connected to one another at their proximal end by means of a bridge section 7. They are made from permanently deformable material, in particular metal, e.g. titanium or a titanium alloy, and the legs 5, 6 can be pressed towards one another out of an open position, in which they are spaced from one another (left ligature clip in FIG. 1), into a closed position, in which the legs are brought closer to one another and close a vessel section 8 arranged between them and are held in clamped position on this vessel section 8 (right ligature clip in FIG. 1).

To deform the ligature clips 4 from the open position into the closed position, the ligature clips 4 are inserted between two spaced clamping jaws 9, 10 of the instrument 1, and these clamping jaws 9, 10 are brought closer to one another, wherein they deform the bridge section 7 of the ligature clips 4 and move the legs 5, 6 substantially parallel towards one another.

The two clamping jaws 9, 10 are located on a structural part, which is arranged in the interior of the barrel 2 in the receiving space with a semicircular cross-section inside the barrel 2, which is located next to the cartridge 3 and is not filled by this.

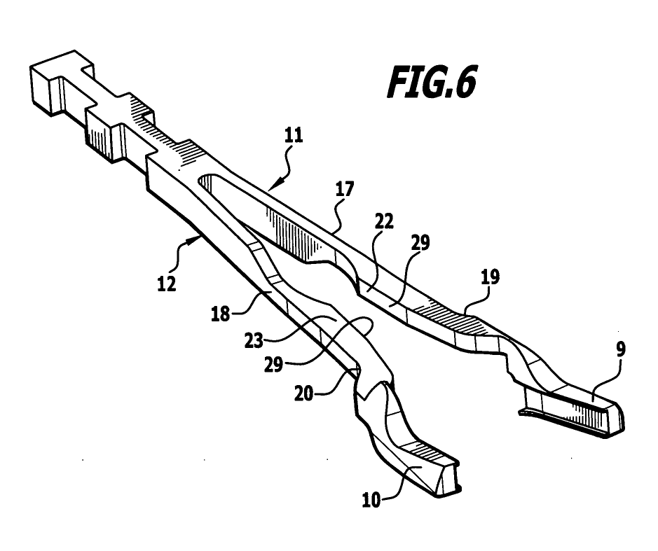

This structural part (FIG. 6) has two arms 11, 12 lying next to one another, which are connected to one another at the proximal end and respectively bear one of the two clamping jaws 9 or 10 at their distal end. The structural part is configured in one piece and is made from an elastically deformable material, in particular from an elastically deformable metal. The arms 11, 12 are thus elastically bendable and can therefore be pivoted elastically relative to one another out of the slightly spread non-deformed position (FIG. 6), so that the two clamping jaws 9, 10 thus approach one another.

The arms 11, 12 are inserted into a chamber 13 of a slide 14 that surrounds the arms over the largest portion of their length. The slide 14 has a semicircular cross-section and completely fills the receiving space of the barrel 2 next to the cartridge 3, so that the slide 14 abuts against the inside wall of the barrel 2 and is longitudinally displaceably guided therein. The chamber 13 has two parallel side walls 15, 16, which extend over the largest part of the length of the slide and are spaced from one another, and the outer surfaces 17, 18 of the arms 11, 12 abut against these side walls 15, 16 when the arms are inserted into the chamber 13. The outer surfaces then run substantially parallel to one another, and in this position the arms 11, 12 are located in a starting position.

The outer surfaces 17, 18 of the arms 11, 12 are widened when the arms 11, 12 exit at the distal end of the slide 14 and there have outwardly sloping abutment surfaces 19, 20, against which the slide 14 abuts with its distal end 21. The two clamping jaws 9, 10 directly adjoin these abutment surfaces 19, 20.

As a result of the distal end 21 of the slide 14 abutting against the abutment surfaces 19, 20, the slide presses the two arms 11, 12 against one another as the slide advances relative to the arms in distal direction, i.e. the arms are pivoted relative to one another by the distal end 21 sliding along on the abutment surfaces 19, 20, wherein the arms 11, 12 are bent elastically inwards. This causes the clamping jaws 9, 10 to move closer, i.e. causes a movement of the clamping jaws from the open position into the closed position.

The displacement between the slide and the arms is achieved by coupling the slide or the arms to one of the transmission elements that pass through the barrel 2, and it is possible to dispose the slide to be longitudinally displaceable in the barrel and to displace it relative to the arms that are connected non-displaceably to the barrel. However, the reverse is also possible: to fix the slide relative to the barrel and thus displace the arms in longitudinal direction. It is merely essential that the arms and the slide are displaced relative to one another in the longitudinal direction, so that the distal end 21 of the slide 14 slides along on the abutment surfaces 19, 20.

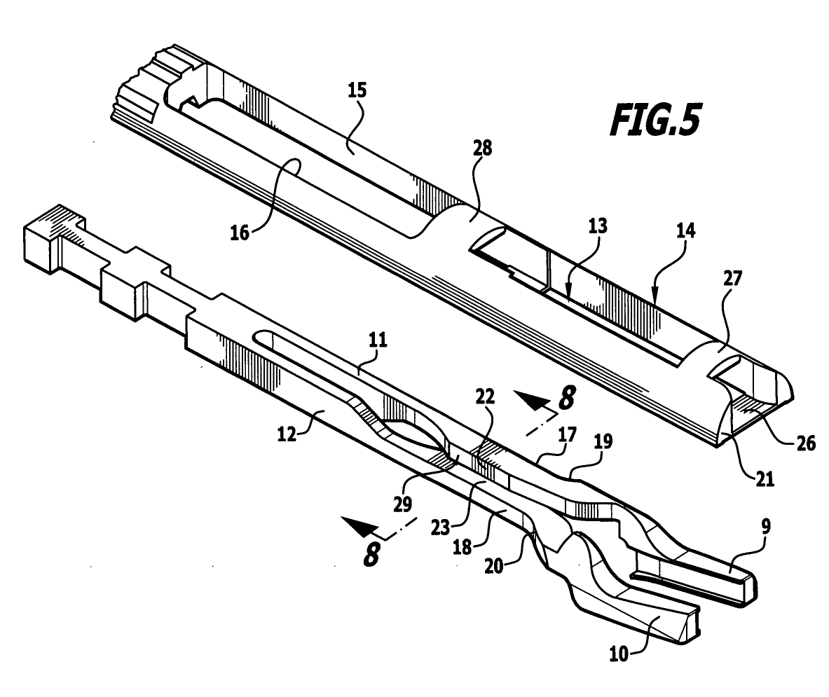

In the interior of the chamber 13 the arms 11, 12 are arranged so that they overlap at least in some sections, as is particularly clearly evident from the illustration in FIG. 5. In the exemplary embodiment shown there, the arms firstly have a constant height starting from the proximal end, the height is reduced approximately to a half at about the centre of the arms, i.e. in such a manner that one arm is located in a lower plane and the other arm is located in an upper plane, which are displaced relative to one another to such an extent that the arms no longer lie next to one another in the region of reduced height but lie one above the other. As a result, it is possible to pivot the arms relative to one another without them hindering one another in their respective movement during this pivoting movement. The overlapping can already occur in the resting position, as shown in FIG. 5, i.e. before the arms are pivoted into the closed position of the clamping jaws, and the overlapping is further increased when the arms pivot into the closed position of the clamping jaws until the overlapping arm regions 22, 23 completely overlap one another, if necessary. As a result of the two arms overlapping the complete width of the chamber 13 is available to each arm during the pivoting movement, so that a relatively large angle range can be covered during the closing movement of the arms.

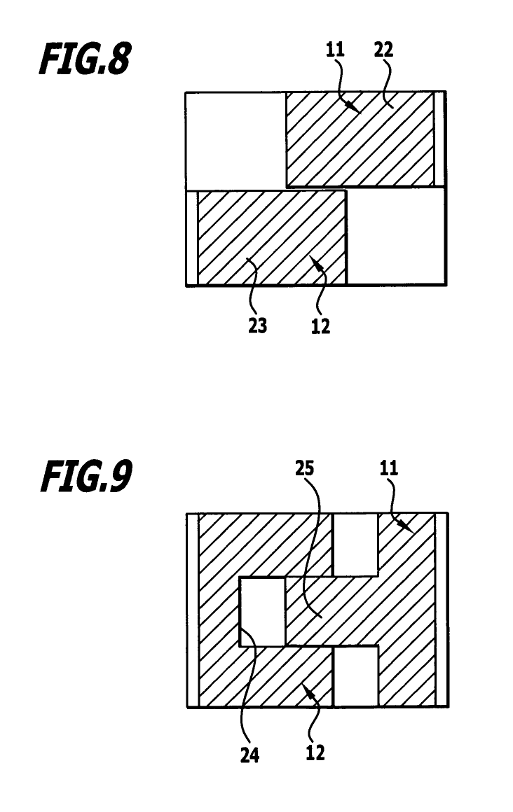

In the exemplary embodiment shown in FIG. 5, the arms are configured wider in the overlapping region 22, 23 than in the proximally adjoining region, in which the arms have full height. This widening is possible because space has been gained in pivoting direction as a result of the arrangement of the arms in different planes, and this widening enables the bending moment of the arms in the overlapping region 22 to be retained or even increased in spite of the reduced height. As evident from the illustration of FIG. 8, the cross-section of the overlapping regions 22, 23 can be a rectangular cross-section, for example, in which the width is larger than the height. The width of the arms, i.e. the extent in pivoting direction, quadratically influences the bending moment of the arms in the respective pivoting plane, while the height influences this only linearly. As a result, the reduction of the bending moment by reduction of the height can be readily compensated or even overcompensated by a relatively small widening of the arms in this region.

In the exemplary embodiment illustrated in FIG. 5, the overlapping of the two arms is only provided over a part-region of the length of the arms, which substantially adjoins the distal end of the arms in the proximal direction, but it would also be possible to allow the arms to overlap over their entire length.

It is essential that the arms do not interfere with one another during the pivoting movement as a result of their displacement in different planes, so that a larger pivoting range is available for both arms.

In the described embodiment, because of the displacement of the two arms in different planes the abutment surfaces 19, 20 are also located in different planes, so that these abutment surfaces 19, 20 also abut against the slide 14 in different planes.

In the configuration of FIG. 5, the overlapping regions are located in different planes, i.e. there is an upper overlapping region of one arm and a lower overlapping region of the other arm, as is also shown in FIG. 8.

It would also be possible to use other cross-sections of the arms, e.g. as shown in FIG. 9, one arm could have a groove-shaped recess 24, into which the second arm engages with a corresponding strip-like projection 25. This also results in an overlapping of the two arms and an increase in the bending moments, but the two arms still do not hinder one another in the overlap region, allowing a greater pivoting ability than in the case of arms that are arranged next to one another without engaging with one another and have corresponding bending moments. The optimum pivoting ability is naturally achieved by a configuration, in which the arms are arranged completely in different planes and therefore have the entire width of the chamber available for their pivoting movement.

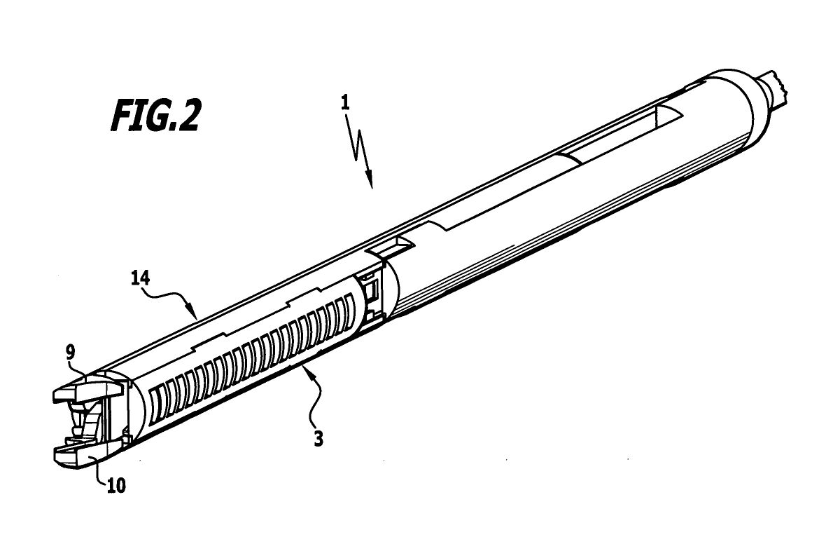

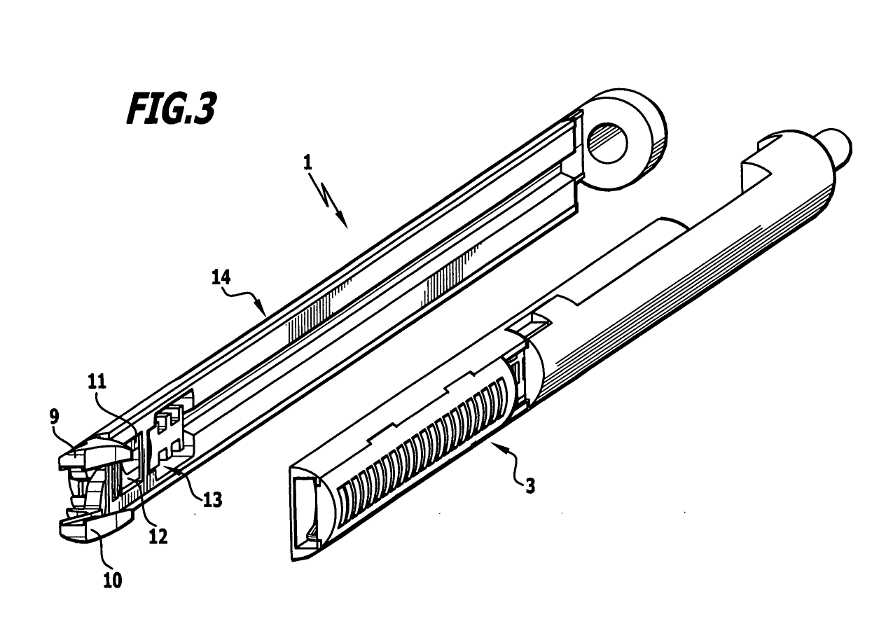

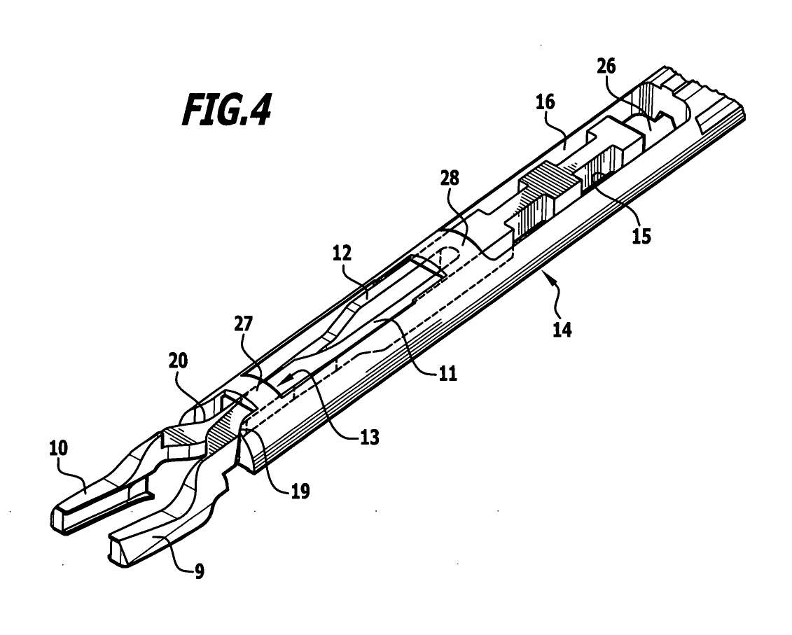

In the exemplary embodiment of FIGS. 1 to 5, the slide 14 receiving the arms 11, 12 is configured so that the slide surrounds the arms on all sides. For this, the chamber 13 has a plane base on its underside that connects the two side walls 15, 16 and webs 27, 28 that connect the side walls 15, 16 on the upper side, i.e. the arms are enveloped by the slide on all sides at least in the region of the webs 27, 28, thus resulting in a relatively high stability of the structural unit consisting of the slide and the arms.

However, in this configuration a part of the cross-section available within the barrel 2 is filled by the side walls 15, 16 and this part of the cross-section is therefore not available for the pivoting movement of the arms 11, 12.

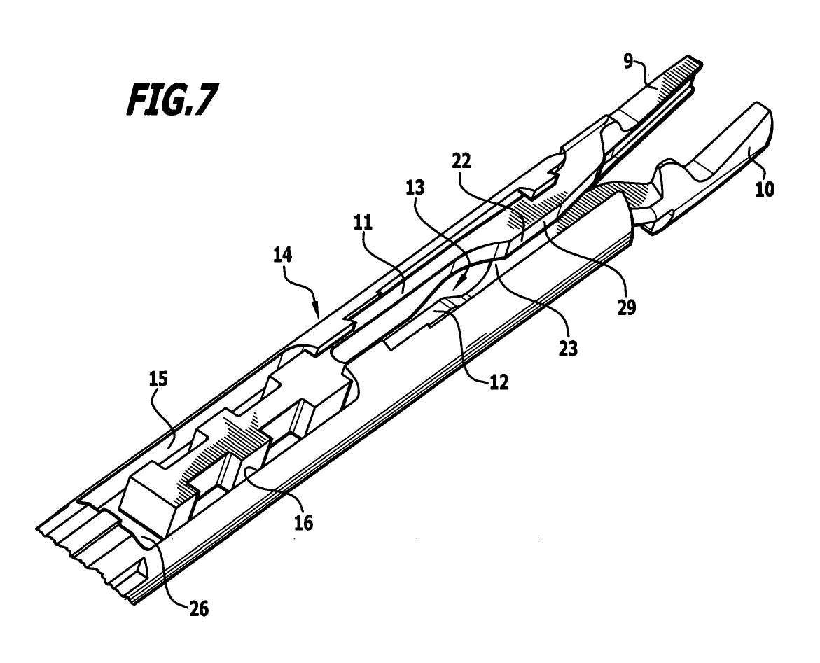

To be able to utilise the full cross-section of the barrel, in the exemplary embodiment of FIG. 7 the slide is no longer configured so that it surrounds the chamber 13 on all sides, but in this exemplary embodiment the webs have been omitted and the height of the side walls 15, 16 is restricted so that there is no material of the slide 14 located opposite the inside faces 29 of the arms 11, 12, i.e. the arms 11, 12 can extend as far as the edge of the semicircular contour defined by the barrel 2. As a result, the pivoting range of the arms is increased, but this also results in a reduction in strength of the slide 14 in the distal region. However, this can be compensated by the slide 14 being supported against the inside face of the barrel 2 at its outer surface and thus being given additional guidance.

1. A surgical instrument for the placement of C-shaped ligature clips having two legs connected to one another by means of a bridge section with two clamping jaws, which in an open position receive an open ligature clip between them and are movable into a closed position by approaching one another and in so doing close the ligature clips by their legs approaching one another, with two elastically deformable arms, each bearing a clamping jaw at its distal end, and with a slide, which is displaceable relative to the arms in the longitudinal direction thereof, and which abuts against the abutment surfaces of the arms and as a result of this abutment against the abutment surfaces elastically pivots the arms relative to one another in a pivoting plane out of a starting position into an end position upon displacement of the slide relative to the arms, wherein at least over a part of their length and at least over a part of their width the two arms are arranged in planes running parallel to the pivoting plane and displaced relative to one another and overlap one another at least partially.

2. An instrument according to claim 1, wherein in the overlap region one of the arms is arranged completely in an upper plane and the other arm in a lower plane.

3. An instrument according to claim 1, wherein in that in the overlap region one of the arms has a recess open towards the other arm, into which recess the other arm projects as least partially.

4. An instrument according to claim 1, wherein the arms already overlap partially in their starting position, in which the clamping jaws are in the open position.

5. An instrument according to claim 1, wherein in their end position, in which the clamping jaws are in the closed position, the arms overlap at least in some areas over their entire width.

6. An instrument according to claim 1, wherein the width of the arms in the overlap region is greater than their height.

7. An instrument according to claim 1, wherein in the starting position the side faces of the arms located on the outside are arranged within a contour with bounding means running parallel to one another over the entire length of the arms with the exception of the distal end region.

8. An instrument according to claim 7, wherein the bounding means is formed by the side walls of a chamber of the slide receiving the arms.

9. An instrument according to claim 1, wherein at its distal end the slide abuts against outwardly projecting abutment surfaces of the arms, which directly adjoin the clamping jaws (9, 10) in proximal direction.

10. An instrument according to claim 9, wherein the abutment surfaces of the arms lie in different planes.

11. An instrument according to claim 9, wherein on one arm the abutment surfaces lie in the plane of the corresponding arm.

12. An instrument according to claim 1, wherein the arms are surrounded on all sides by the slide at least on a part of their length.

13. An instrument according to claim 1, wherein the overlap region no part of the slide lies opposite the side faces of the arms located on the inside.

14. An instrument according to claim 1, wherein in the non-deformed state the arms do not overlap over their entire length.

15. An instrument according to claim 1, wherein the arms intersect between their proximal end and their distal end.

16. An instrument according to claim 1, wherein the arms and the slide are arranged together inside a tubular barrel with a circular contour, beyond which the clamping jaws likewise do not project in their open position.
